(19) United States
(12) Patent Application Publication
(10) Pub. No.: US 2025/0332581 A1
(43) Pub. Date: Oct. 30, 2025

(54) ARTIFICIAL PHOTOSYNTHESIS USING TITANIUM, ZIRCONIUM, AND HAFNIUM TETRAHALIDE COMPLEXES WITH VISIBLE-LIGHT-ACTIVE CHROMOPHORES, INCLUDING 2-PHENYL INDOLE AND 2-PHENYL BENZOXAZOLE

(71) Applicant: Gregory G. Arzoumanidis, Naperville, IL (US)

(72) Inventor: Gregory G. Arzoumanidis, Naperville, IL (US)

(21) Appl. No.: 19/083,408

(22) Filed: Mar. 18, 2025

Related U.S. Application Data
(60) Provisional application No. 63/566,874, filed on Mar. 18, 2024.

(51) Int. Cl. B01J 31/18; B01J 35/39; C01B 3/04; C07C 51/15

(52) U.S. Cl. CPC B01J 31/1825; B01J 35/39; C01B 3/042; C07C 51/15; B01J 2231/005; B01J 2231/70; B01J 2531/46

(57) ABSTRACT

A novel method for artificial photosynthesis utilizing a chemical system that operates by harnessing visible light and directly capturing carbon dioxide and water from the atmosphere. The system is based on self-organizing complexes comprising visible-light-sensitive chromophores, such as 2-phenyl indole and 2-phenyl benzoxazole, along with titanium tetrachloride, which autonomously perform continuous, complex chemical operations to produce long-chain (C₂ to C₁₇) oxygenated organic materials. The process employs earth-abundant metal coordination compounds, including titanium (Ti), zirconium (Zr), hafnium (Hf), and vanadium (V), in the solid state. These compounds form carbonated metal derivatives upon hydrolysis, which are subsequently reduced through proton transfer from water. The system initially generates C₁ materials that oligomerize via a novel self-catalyzed mechanism intrinsic to the system's operation. Monitoring and characterization of the chemical transformations have been conducted using high-resolution MALDI-TOF (matrix-assisted laser desorption ionization-time of flight) mass spectrometry, supported by infrared (IR) spectroscopy and nuclear magnetic resonance (NMR) analysis. This innovation represents a sustainable and scalable approach for converting atmospheric CO₂ into valuable organic materials.

### CROSS-REFERENCE TO RELATED APPLICATION

This application claims priority from U.S. Prov. Patent App. No. 63/566,874 entitled “ARTIFICIAL PHOTOSYNTHESIS WITH TITANIUM, ZIRCONIUM AND HAFNIUM OXYHALIDES/2-PHENYL INDOLE PHOTOCATALYTIC COMPLEXES”, filed Mar. 18, 2024, the entire disclosure of which is hereby incorporated by reference in its entirety.

### BACKGROUND OF THE DISCLOSURE

### 1. Field of the Disclosure

The present invention relates to the field of artificial photosynthesis (AP), specifically involving visible-light photocatalytic complexes of titanium, zirconium, and hafnium tetrahalides in combination with 2-phenyl indole and related chromophores. These complexes can directly capture atmospheric carbon dioxide (CO₂) and convert it into oxygenated hydrocarbons with linear carbon chains ranging from C₁ to C₂₀. Additionally, the photocatalytic system absorbs atmospheric humidity, facilitating the splitting of water (H₂O) into hydrogen, which is utilized in the reduction of CO₂, as well as the formation of hydrogen peroxide (H₂O₂).

### 2. Background Art

“Living” chemical systems autonomously self-organize, perform complex tasks, and harvest energy from inexhaustible sources like sunlight, air, and water. Such systems, particularly photocatalysts operating away from equilibrium under constant energy and material input, embody artificial photosynthesis's fundamental concept and goal (AP). Inspired by natural photosynthesis, AP seeks to create efficient systems that produce valuable products while offering immense environmental, energy, and economic benefits.

Photocatalytic water splitting, using catalysts such as titanium dioxide (TiO₂), has been extensively studied due to TiO₂'s stability, low cost, and eco-friendliness. However, its efficiency requires significant improvements to achieve commercial viability. Current hydrogen production predominantly relies on indirect methods like solar-driven electrolysis. Meanwhile, direct air capture (DAC) of CO₂ remains energy-intensive, often requiring chemical reactants and high energy input. No reported system-apart from natural photosynthesis-simultaneously captures and reduces CO₂ directly from the atmosphere under ambient conditions. Existing CO₂ reduction methods typically require concentrated CO₂ sources and complex devices, limiting scalability and sustainability.

A more sustainable approach would integrate water splitting and CO₂ reduction into a single, continuous process to produce valuable organic compounds beyond simple molecules like CO, CH₄, or CH₃OH. Current catalytic methods are hindered by complex setups, high energy requirements, and poor reproducibility. Despite decades of research on TiO₂, molecular-level titanium photocatalysts remain underexplored. While surface-bound functionalities like Ti═O, Ti—O—O, and Ti(OH)₂ enhance bulk TiO₂ photocatalysis, molecular titanium complexes, such as titanium oxide dichloride (Cl₂Ti═O), offer a promising avenue for improving photocatalytic efficiency through ligand-to-metal charge transfer (LMCT) photochemistry.

Titanium-, zirconium-, and hafnium-based coordination compounds are abundant, stable, and well-suited for photocatalytic applications. When paired with visible-light-sensitive chromophores, such as 2-phenyl indole and 2-phenyl benzoxazole, these metal halide complexes can form self-organizing systems that harvest light energy and facilitate complex chemical reactions. However, their full potential for autonomous CO₂ capture and conversion under ambient conditions has not yet been realized.

This invention introduces a novel artificial photosynthesis system utilizing titanium, zirconium, and hafnium tetrahalide complexes with visible-light-active chromophores. Operating under ambient conditions, the system directly captures atmospheric CO₂ and water vapor, converting them into long-chain oxygenated hydrocarbons (C₁ to C₂₀) and producing hydrogen peroxide (H₂O₂) as a valuable byproduct. This self-sustaining process leverages a novel self-catalyzed oligomerization mechanism to create complex organic products, representing a significant advancement in green chemistry and renewable energy technologies.

### SUMMARY OF THE DISCLOSURE

The present invention provides a groundbreaking method for artificial photosynthesis, harnessing visible light to directly capture atmospheric carbon dioxide (CO₂) and water (humidity) under ambient conditions. This process utilizes self-organizing titanium, zirconium, and hafnium tetrahalide complexes in combination with visible-light-active chromophores, such as 2-phenyl indole and 2-phenyl benzoxazole, which together form a robust photocatalytic system. The system performs continuous, autonomous chemical operations, converting CO₂ into long-chain oxygenated hydrocarbons with carbon chains ranging from C₁ to C₂₀. These hydrocarbons have significant potential in renewable energy applications, as well as in the production of specialty chemicals and biofuels.

In addition to CO₂ capture and conversion, the photocatalytic system absorbs atmospheric humidity, splitting water (H₂O) into hydrogen, which is subsequently used to reduce CO₂, as well as generating hydrogen peroxide (H₂O₂) as a byproduct. This dual function—simultaneously reducing CO₂ and splitting water—enhances the efficiency of the system, making it highly effective for energy and environmental applications.

The process operates through a novel, self-catalyzed oligomerization mechanism that allows initially produced C₁ materials to oligomerize into larger organic molecules. This system's self-organizing behavior distinguishes it from traditional catalytic systems by enabling continuous product formation without the need for external catalysts or interventions. The products formed in the system include linear hydrocarbons, ranging from simple C₁ units to complex long-chain organic molecules, expanding the range of potential applications for artificial photosynthesis technologies.

The chemical transformations within the system have been characterized using high-resolution MALDI-TOF (matrix-assisted laser desorption ionization-time of flight) mass spectrometry, which enables accurate identification of molecular structures and product distribution. Complementary infrared (IR) and nuclear magnetic resonance (NMR) spectroscopy are used to confirm the identities and purity of the generated compounds, ensuring the reliability of the system's output.

To provide a clearer understanding of the invention, reference is made to the accompanying drawings and detailed description. These illustrate the structural and operational aspects of the self-organizing artificial photosynthesis system, highlighting key components such as the 2-phenyl indole (PI) or 2-phenyl benzoxazole (BX)/titanium tetrachloride complexes, the sequential chemical transformations, and the pathways leading to the formation of long-chain oxygenated hydrocarbons. Together, these sections offer a comprehensive overview of the invention's mechanisms and outcomes.”

### BRIEF DESCRIPTION OF THE DRAWINGS

The invention is further illustrated in the following figures and tables, which provide detailed analytical insights into the described artificial photosynthesis process:

FIG. 1. Mass of complex (PI)2TiCl4 after daylight exposure for about three weeks. The color changes from dark brown to khaki and partially green, as shown.

FIG. 2. First MALDI-TOF spectrum of complex (PI)2TiCl4 in THF, after room-light exposure

FIG. 3. After room-light exposure, the Second MALDI-TOF spectrum of complex (PI)2TiCl4 in THF.

FIG. 4. Third MALDI-TOF spectrum of complex (PI)2TiCl4 in THF, after room-light exposure. PI oligomers recorded.

FIG. 5. MALDI-TOF spectrum of complex (PI)2TiCl4 in CH2Cl2, after room-light exposure.

FIG. 6. FTIR of the mixture of the (PI)2TiCl4 complex after 3 weeks of visible light exposure. Peaks are identified in the spectrum, with their corresponding transmittal values.

FIG. 7. 1H NMR of complex (PI)2TiCl4 after exposure to visible light and air.

FIG. 8. First MALDI-TOF of complex (BZ)2TiCl4 three days after preparation.

FIG. 9. Second MALDI-TOF of complex (BZ)2TiCl4 three days after preparation.

FIG. 10. Third MALDI-TOF of complex (BZ)2TiCl4 three days after preparation.

FIG. 11. MALDI-TOF of complex (BZ)2TiCl4 after 3.5 months, without direct light exposure.

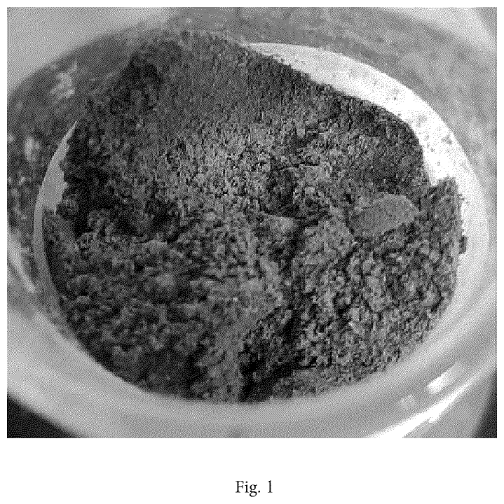

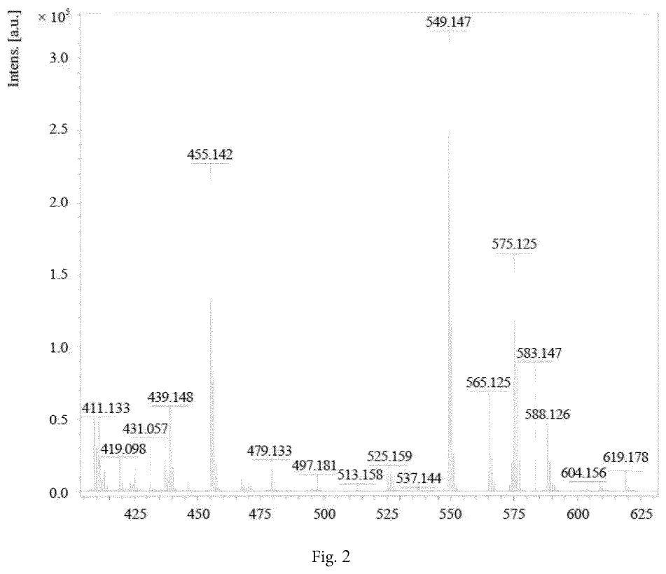

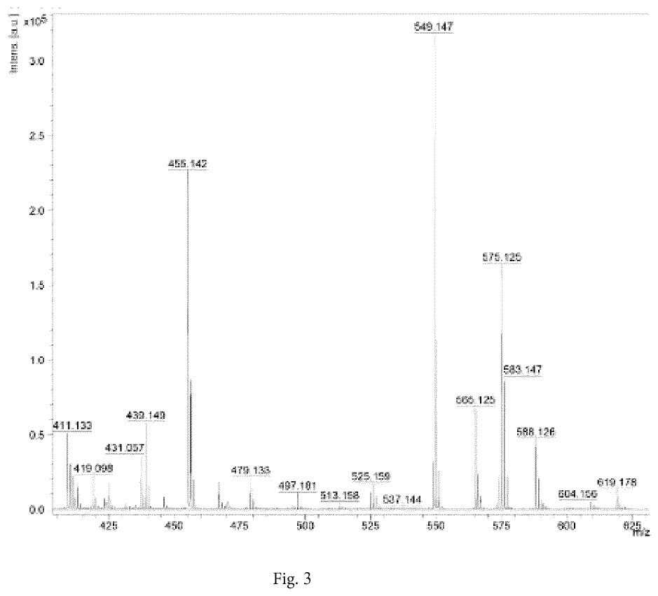

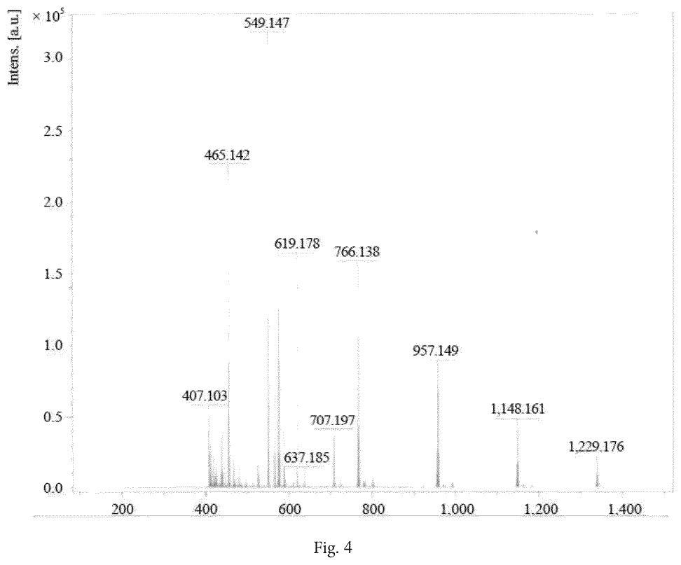

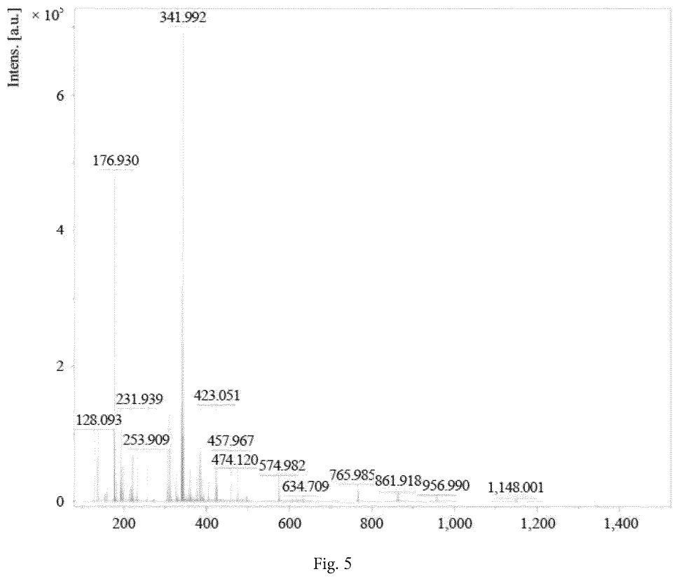

Table 1. Assignments of products in MALDI-TOF of FIG. 5

Table 2. Water-soluble organic products after exposure of (PI)xTiCl4 complexes

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

This invention relates to artificial photosynthesis catalyzed by titanium complexes incorporating photoactive ligands such as 2-phenyl indole (PI) and 2-phenyl benzoxazole. The system operates under visible light and ambient conditions, performing direct atmospheric capture (DAC) of CO₂, splitting water, and producing oxygenated organic compounds, including long-chain hydrocarbons. The synergy between titanium's redox-active coordination sphere and the photoionizable ligands enables a self-sustaining catalytic cycle. Key processes include hydrolysis, CO₂ capture, light-induced redox transformations, and product synthesis.

The catalytic system initiates when Ti—Cl bonds in the parent complex (1) are hydrolyzed by air humidity, forming Ti—OH (2) or transient Ti═O bonds (3, 4).

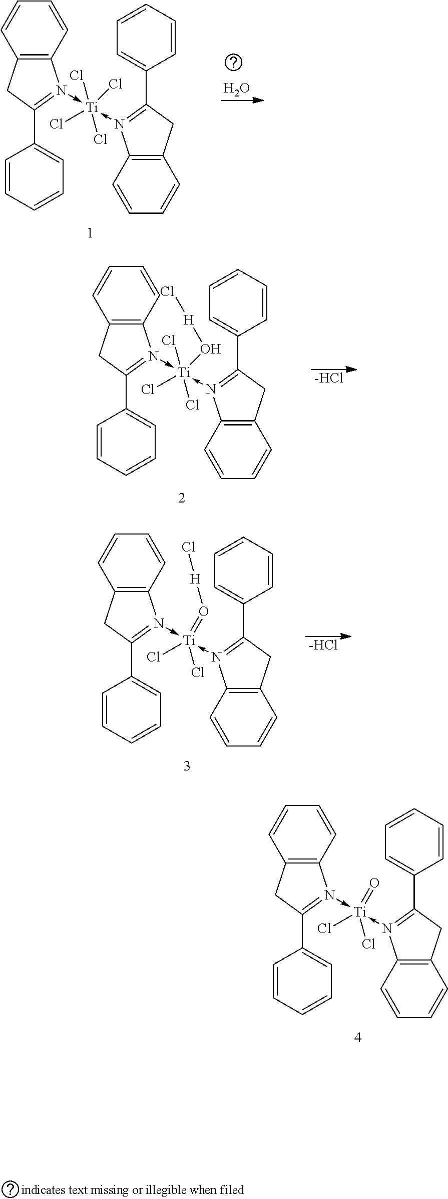

These functionalities are key to enabling the visible light photocatalytic cycle. A crucial intermediate, complex 4, forms through this hydrolysis and may also be prepared directly by reacting two equivalents of PI with TiOCl₂.

Upon visible light irradiation, Ti—OH expels OH radicals, reducing Ti(IV) to Ti(III) and forming complex 44.

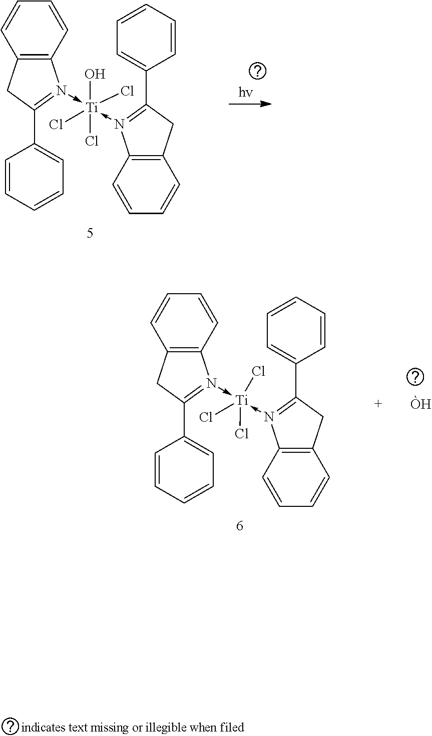

Chlorine radicals, derived from HCl interaction, contribute to para-chlorination of the PI ligand. Further hydrolysis leads to complex 45, which transforms under light to the transient Ti(II) species 46, enabling DAC of CO₂ and interactions with small molecules such as CH₂O, CO, and O₂.

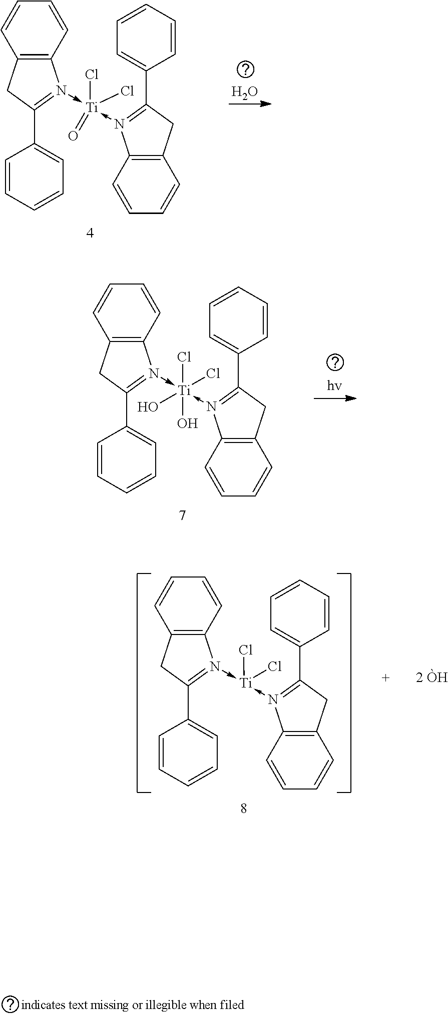

The system autonomously captures and reduces atmospheric CO₂ under ambient conditions. Complex 6 reacts with CO₂, forming carbonate-containing intermediates such as complex 30 and 31.

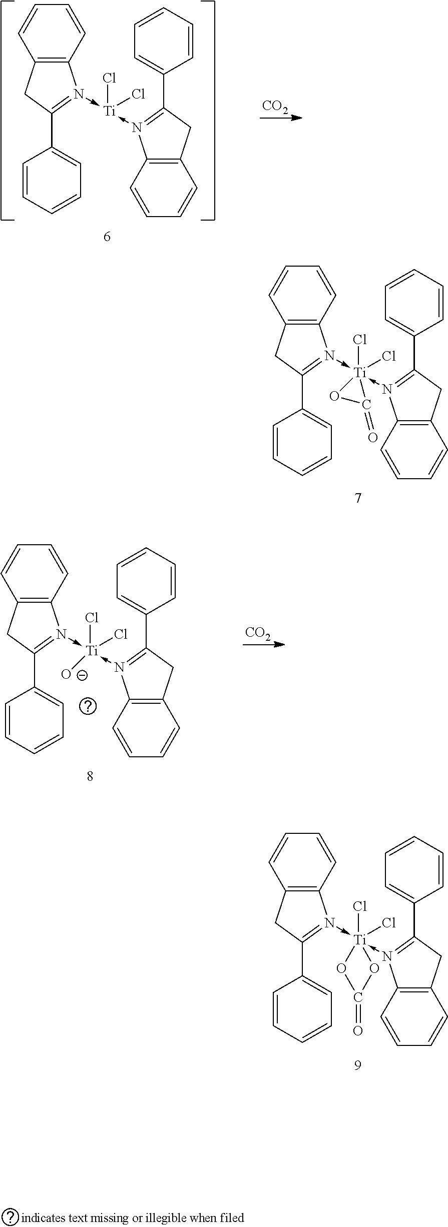

This process is one of the first examples of a mononuclear titanium complex interacting with atmospheric CO₂ to form cyclic carbonate groups. These intermediates are precursors to organic product synthesis and highlight the system's potential for sustainable CO₂ utilization.

Captured CO₂ is reduced to CO, HCHO, and CH₃OH using system-generated H₂ or via hydrogen transfer from water.

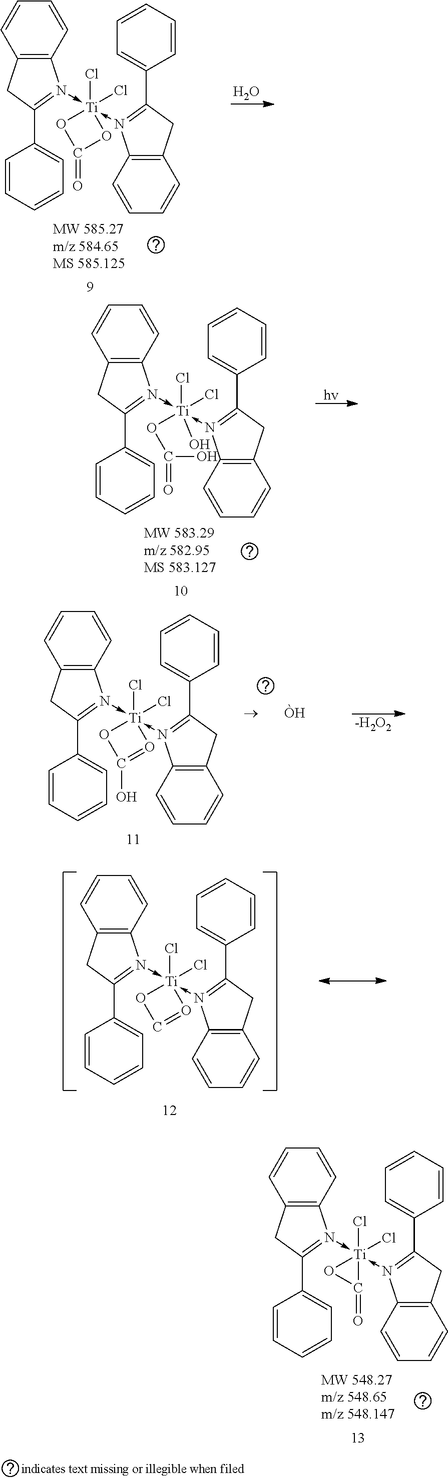

Formaldehyde and methanol couple photocatalytically to form ethylene glycol (HOCH₂CH₂OH), which integrates into titanium complexes.

The catalytic system's ability to utilize DAC-derived CO₂ for synthesizing oxygenated organics showcases its potential for large-scale sustainable chemical production.

The system extends DAC-reduced formaldehyde into long-chain oxygenated hydrocarbons.

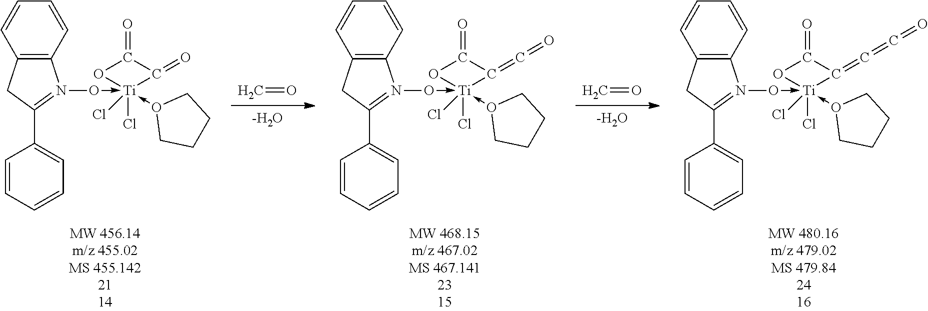

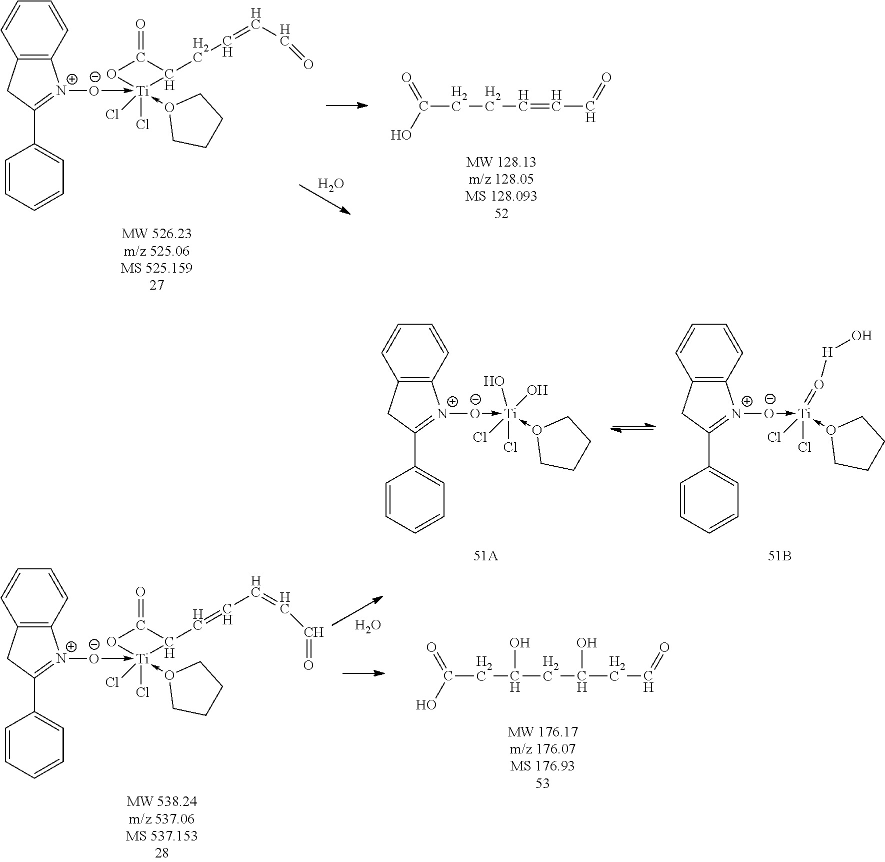

Titanium α-ketocarboxylate complex 21 catalyzes a dehydration synthesis of formaldehyde, forming ketene-containing intermediates (complex 23). Subsequent chain growth produces C2-C6 products with alpha-omega functionalities, mimicking natural photosynthetic pathways.

Ligand exchange involving PI and donor molecules (e.g., THF, HCHO) results in mixed-donor complexes.

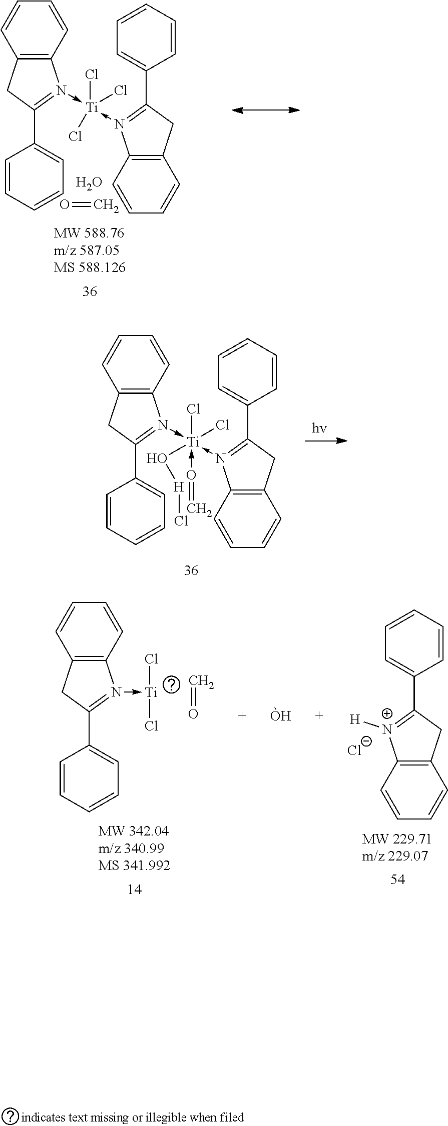

These exchanges influence the overall catalytic efficiency by providing open coordination sites on titanium for system-generated small molecules, ensuring active participation in the reaction cycle.

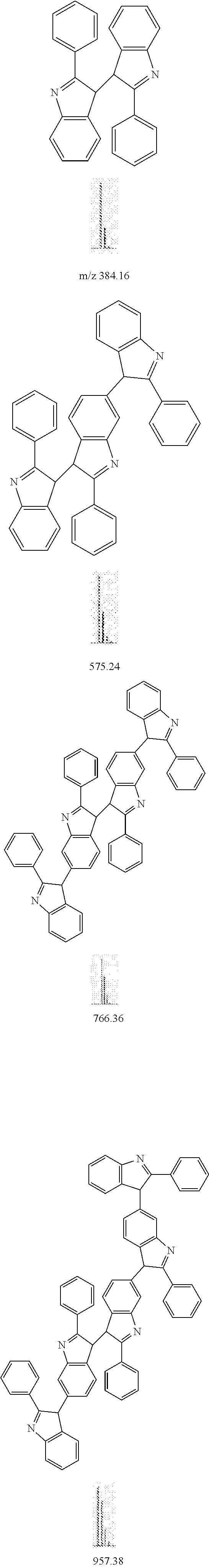 [SMILES: c1ccc(C2=Nc3ccccc3C2C2C(c3ccccc3)=Nc3ccccc32)cc1 ; c1ccc(C2=Nc3ccccc3C2c2ccc3c(c2)N=C(c2ccccc2)C3C2C(c3ccccc3)=Nc3ccccc32)cc1 ; c1ccc(C2=Nc3ccccc3C2c2ccc3c(c2)N=C(c2ccccc2)C3C2C(c3ccccc3)=Nc3cc(C4C(c5ccccc5)=Nc5ccccc54)ccc32)cc1 ; c1ccc(C2=Nc3ccccc3C2c2ccc3c(c2)N=C(c2ccccc2)C3c2ccc3c(c2)N=C(c2ccccc2)C3C2C(c3ccccc3)=Nc3cc(C4C(c5ccccc5)=Nc5ccccc54)ccc32)cc1]

Photoionized PI radicals undergo oligomerization. This process consumes PI ligands from titanium complexes, forming oligomers detected via MALDI-TOF. The implications of these oligomers on system performance and their potential electronic and fluorescent properties remain subjects for further study.

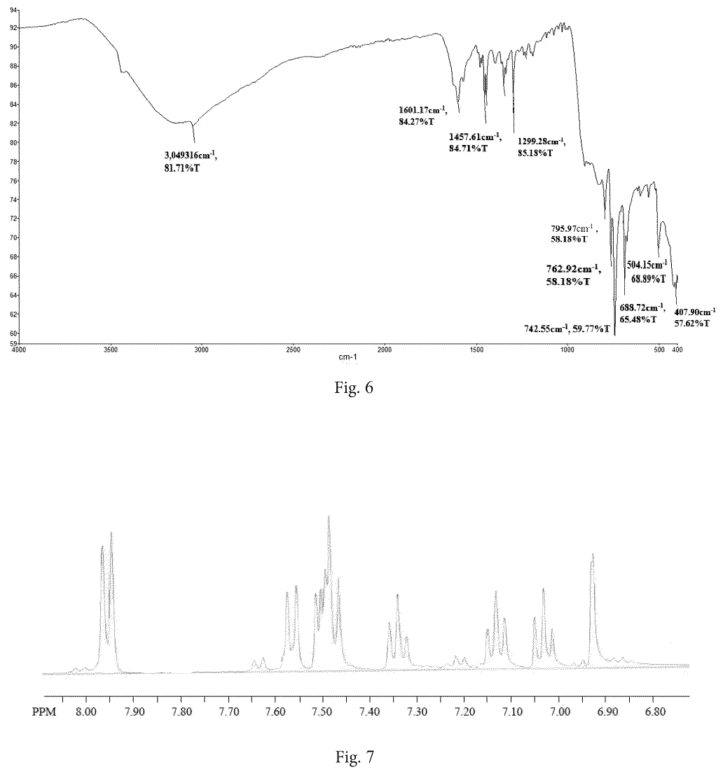

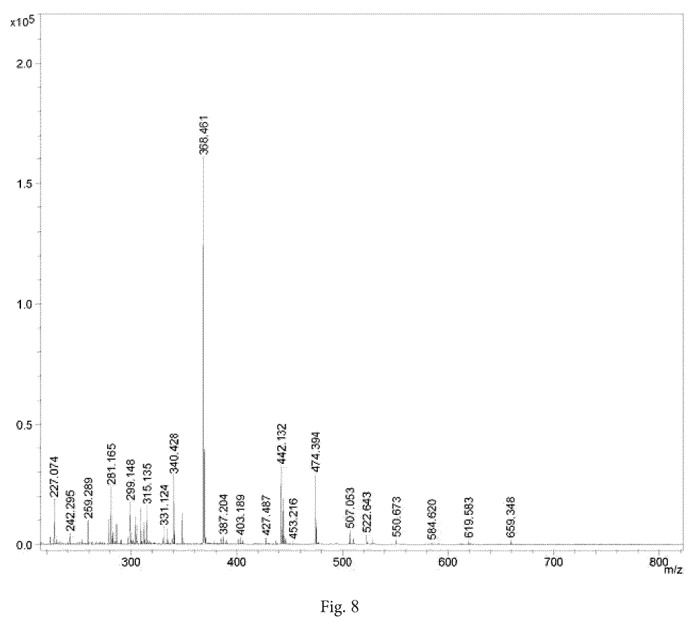

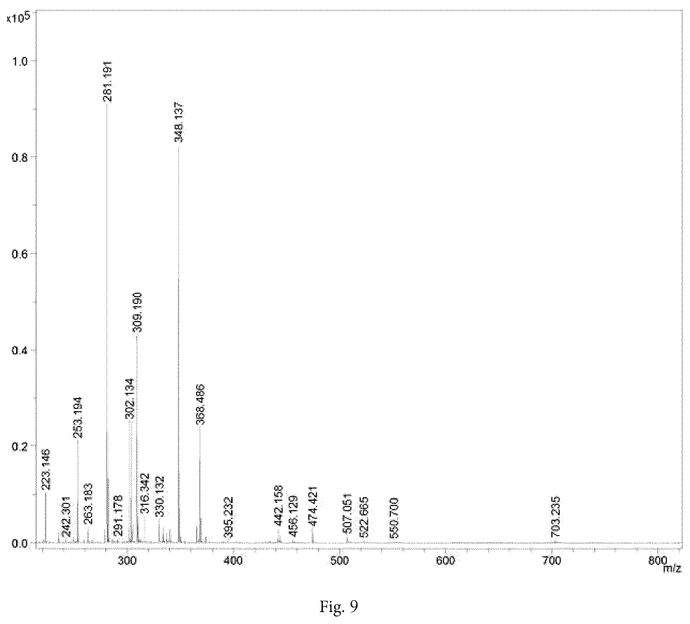

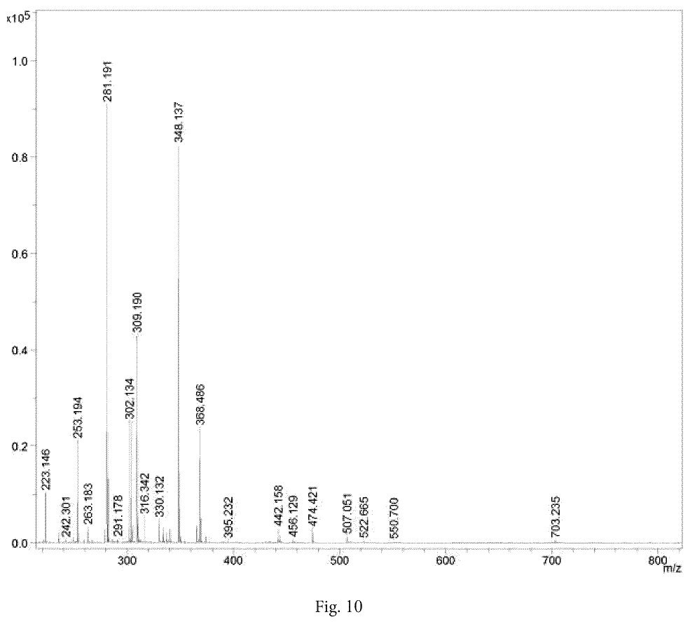

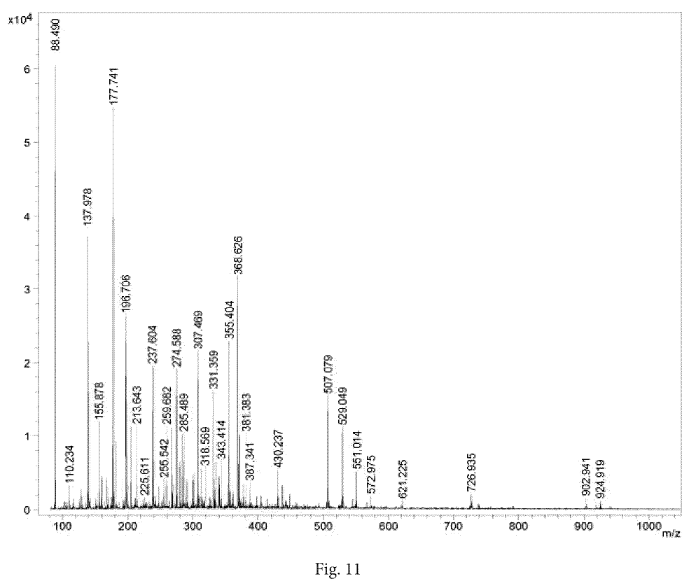

Hydrolysis products from 2:1, 1:1, and 1:2 PI/TiCl4 complexes exposed to visible light: To optimize the molar ratio of PI/TiCl4 complexes for improved product yield and distribution, we subjected complexes prepared at PI:TiCl4 molar ratios of 2:1, 1:1, and 1:2 to hot water treatment. These were exposed to visible light under the same conditions as the 2:1 complex (see FIG. 6). soluble and insoluble fractions were observed in all cases. MALDI-TOF analysis was performed on each fraction, with the results of the water-soluble fractions summarized in Table 1.

Table 1 presents the MALDI-TOF data in the first column, the proposed chemical product structures in the second, and the relative product distributions (on a 0-8 scale) for each complex in the subsequent three columns. The calculated molecular weights (MW) and m/z values align well with the experimentally obtained MS data for all proposed products. Notably, most products are consistent across the three complexes, highlighting the system's reproducibility. The most compelling results were observed for the 1:1 complex, which exhibited a reduced product diversity, higher overall relative yield, and a dominant product at m/z 177.0431, accounting for over 40% of the total yield. This suggests that the molar ratio in the complex significantly influences the chemical product distribution—a crucial feature of this catalytic system that warrants further investigation.

A unique peak at m/z 507.0319 was observed exclusively in the 1:1 complex. This peak is tentatively assigned to the coupling product of a C9 oxygenated species with a

TABLE 1
Relative MALDI-TOF Intensity of water-soluble products from visble light exposed PI/TICI, Complexes
Relative MALDI-TOF Intensity of water-soluble products from visble light exposed PI/TICI, Complexes

| MALDI-TOF | | | PI:TiCl₄ Molar Ratio | | |
|---|---|---|---|---|---|
| DATA | | Products | 2:1 | 1:1 | 1:2 |
| MW m/z MS | 80.87 80.95 80.9925 | 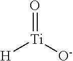 | 1.05 | 3.1 | 0.43 |
| MW m/z MS | 96.87 96.94 96.9557 | 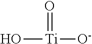 | 0.5 | 1.4 | 0.2 |
| MW m/z MS | 126.11 126.03 126.1728 | 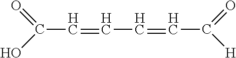 | 0.41 | | 1.4 |
| MW m/z MS | 128.12 128.05 128.1728 | 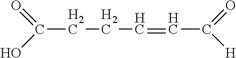 | 0.41 | | 1.4 |
| MW m/z MS | 130.14 130.06 130 | 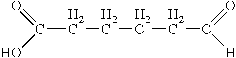 | 0.41 | | 0.5 |
| MW m/z MS | 138.12 138.03 137.0377 | 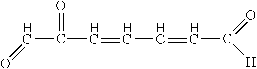 | 2.05 | 3.8 | 1.95 |
| MW m/z MS | 159.21 159.10 159.0284 | 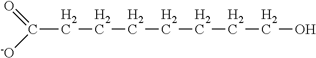 | 1.80 | 1.80 | 2.82 |
| MW m/z MS | 176.17 176.07 177.0431 | 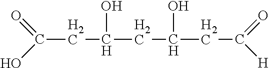 [SMILES: O=CCC(O)CC(O)CC(=O)O] | 0.5 | 7.9 | 3.22 |
| MW m/z MS | 199.18 199.06 199.0083 | 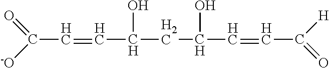 | 0.16 | 1.0 | 0.15 |
| MW m/z MS | 311.22 311.08 311.2125 | 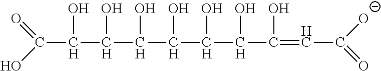 [SMILES: O=C([O-])C=C(O)C(O)C(O)C(O)C(O)C(O)C(O)C(=O)O] | 4.2 | | 0.88 |
| MW m/z MS | 397.31 397.10 387.1651 | 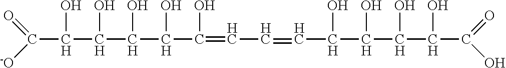 | 0.15 | 8.5 | 0.48 |
| | | Relative Total Amounts | 11.26 | 19.50 | 13.53 |

chlorinated PI ligand (Arzoumanidis et al. 2024). Additionally, a geometric projection based on total product yields across the three complexes suggests an optimal PI:TiCl4 molar ratio of approximately 1.25, with an estimated relative product yield of 25 (see Table 1).

The hot water-insoluble fraction primarily consisted of PI oligomers, forming a continuous series with decreasing concentrations, containing up to 10 monomers (see FIG. 28). A minor sequence of m/z peaks was also observed, increased by 34 m/z units relative to the primary sequence (e.g., 418-384, 609-575, 800-766, 991-957). In each pair, the higher value corresponds to a PI oligomer incorporating a chloride substitution.

The described photocatalytic system integrates DAC of CO₂, water splitting, and production of valuable oxygenated organics through a series of light-driven transformations. Titanium's redox flexibility, combined with the photoactivity of PI ligands, creates a sustainable platform for artificial photosynthesis with significant environmental and economic implications.

### DETAILED DESCRIPTION OF THE INVENTION

Introduction This invention relates to the field of artificial photosynthesis and chemical catalysis, specifically the utilization of earth-abundant titanium coordination complexes to produce long-chain oxygenated organic compounds from atmospheric CO2 and water under ambient conditions. The invention leverages a self-organized, autocatalytic system energized by visible light, demonstrating unprecedented efficiency and scalability in carbon fixation and conversion.

Overview of the System The invention employs 2-phenyl indole (PI) complexes of titanium tetrachloride, represented as (PI)2TiCl4, as primary catalytic units. These complexes, when exposed to visible light, undergo hydrolysis by atmospheric humidity, forming organotitanium intermediates. These intermediates capture and reduce CO2 directly from ambient air, producing C2 to C17 oxygenated hydrocarbons.

### Mechanism of Action

3.1 Initial Activation: Under illumination, the (PI)2TiCl4 complex absorbs visible light, leading to the excitation of the photoactive ligand (PI). This excitation facilitates charge transfer from the ligand to the titanium center, increasing its reactivity. Hydrolysis of the Ti—Cl bonds occurs due to ambient moisture, forming Ti—OH intermediates.
3.2 CO2 Capture and Reduction: The Ti—OH intermediates interact with atmospheric CO₂ to form carbonate-like species, which are further reduced via proton transfer from water. This reduction results in the formation of C1 compounds, primarily formaldehyde.
3.3 Autocatalytic Chain Growth: Formaldehyde acts as a feedstock for subsequent autocatalytic reactions within the system. Through a combination of aldol-like condensations and radical-mediated processes, these initial products oligomerize to form higher oxygenated hydrocarbons, ranging from C6 to C17. Hydroxyl radicals generated during the photocatalytic cycle play a critical role in initiating and propagating these chain growth reactions.
4. Synergistic Role of Hydroxyl Radicals A key aspect of the invention is the synergistic interaction between hydroxyl radicals and organotitanium intermediates. Hydroxyl radicals, generated through the photoinduced cleavage of Ti—OH bonds, exhibit high reactivity, facilitating:
   Hydrogen abstraction from intermediates to generate radicals.
   Initiation of radical chain reactions leading to dimerization and higher oligomer formation.
   Selective oxidation, enabling the formation of functionalized organic products.
5. Product Distribution and Versatility The system predominantly produces α-carboxylic acid-ω-aldehyde compounds within the C6-C9 range, which can further undergo dimerization to yield C10-C17 hydrocarbons. MALDI-TOF mass spectrometry confirms the molecular weight distribution, and IR and NMR analyses validate the structures of the products. The ability to control product distribution by varying parameters such as light intensity, PI:TiCl4 molar ratio, and reaction conditions underscores the versatility of this system.
6. Advantages over Existing Technologies This invention represents a significant advancement over existing artificial photosynthesis technologies due to:
   Scalability: Direct utilization of atmospheric CO₂ and water eliminates the need for concentrated feedstocks.
   Efficiency: Visible-light activation reduces energy requirements compared to traditional methods.
   Sustainability: Use of earth-abundant titanium complexes ensures environmental and economic viability.
   Product Range: Capability to produce long-chain oxygenated hydrocarbons with high selectivity and yield.

### EXAMPLES

Example 1: Preparation of (PI)2TiCl4 Complex 2-Phenyl indole (2 mmol) was dissolved in dichloromethane (10 mL), and TiCl4 (1 mmol) was added dropwise under nitrogen atmosphere. The mixture was stirred at room temperature for 2 hours, resulting in the formation of (PI)2TiCl4 as a yellow solid. The product was isolated by filtration and characterized using NMR and IR spectroscopy.

Example 2: Photocatalytic Reaction under Ambient Conditions The (PI)2TiCl4 complex (50 mg) was dispersed in water (10 mL) and exposed to visible light (400-700 nm) in an open-air setup. MALDI-TOF analysis of the reaction mixture after 24 hours revealed the formation of C6-C9 α-carboxylic acid-ω-aldehyde compounds.

Example 3: Chain Growth to C17 Hydrocarbons To a solution of the initial reaction products, additional formaldehyde (1 mmol) was introduced, and the mixture was irradiated for 48 hours. Mass spectrometry confirmed the formation of dimerized and oligomerized products up to C17.
8. Applications The invention has broad applications, including:
   Sustainable production of biofuels and value-added chemicals.
   Carbon capture and utilization technologies for mitigating climate change.
   Photochemical systems for renewable energy storage and conversion.
9. Conclusion This invention demonstrates a novel approach to artificial photosynthesis, utilizing a self-organized titanium-based catalytic system to convert atmospheric CO2 and water into valuable organic materials. The innovative mechanism, high efficiency, and environmental sustainability make it a transformative technology in the field of renewable energy and green chemistry.

What is claimed is:

1. A catalytic system for artificial photosynthesis, comprising:
A coordination complex of the general formula LxMY4, wherein L is a photochemically active ligand capable of absorbing visible light and facilitating charge transfer, x is any number from 0.5 to 2, M is a transition metal selected from titanium (Ti), zirconium (Zr), hafnium (Hf), or vanadium (V), Y is a halogen or pseudohalogen selected from chloride, bromide, fluoride, cyanide, or thiocyanate;
A mechanism for hydrolyzing the complex under ambient humidity to produce reactive intermediates; and
A visible-light-driven catalytic process for the conversion of atmospheric CO₂ and water into oxygenated organic compounds ranging from C2 to C17.

2. A method for producing long-chain oxygenated hydrocarbons via artificial photosynthesis, the method comprising:
Providing a catalytic complex of the formula LxMY4, where L, x, M, and Y are as defined in claim 1;
Activating the complex with visible light to generate excited states and reactive intermediates;
Hydrolyzing the complex in the presence of water to form metal hydroxyl species; and
Reducing CO₂ captured from ambient air via radical-mediated reactions to produce oxygenated hydrocarbons in the range of C2 to C17.

3. The catalytic system of claim 1, wherein L is selected from heterocyclic aromatic ligands substituted with electron-donating or electron-withdrawing groups to tune the absorption spectrum and photocatalytic performance.

4. The catalytic system of claim 1, wherein MMM is titanium, and the ligand L is 2-phenyl indole or 2-phenyl benzoxazole.

5. The catalytic system of claim 1, wherein the ratio of L:M is adjusted to control product selectivity, favoring either lower hydrocarbons (C2 to C5) or higher hydrocarbons (C6 to C17).

6. The method of claim 2, wherein the visible light activation occurs at wavelengths between 450 nm and 650 nm, optimizing energy absorption for photocatalysis.

7. The method of claim 2, further comprising the step of oligomerizing lower hydrocarbons produced in the reaction to yield higher oxygenated hydrocarbons.

8. The catalytic system of claim 1, wherein YYY includes pseudohalogens such as cyanide or isocyanate, enhancing the catalytic activity and stability of the complex.

9. The method of claim 2, wherein the process simultaneously generates hydrogen gas as a byproduct during the reduction of water and CO₂.

10. The catalytic system of claim 1, wherein L comprises a combination of two or more photochemically active ligands with complementary light absorption properties.

11. The method of claim 2, wherein the hydrolysis of the complex produces intermediates of the formula LxM(OH)nY4-n, where n=1, 2, or 3, facilitating multi-step reduction pathways.

12. The catalytic system of claim 1, further comprising an auxiliary co-catalyst or stabilizer to enhance product yield and minimize deactivation of the primary catalytic complex.

13. The method of claim 2, wherein the oxygenated hydrocarbons include alpha-carboxylic acid-omega-aldehyde derivatives that can undergo further chemical transformation.

14. The catalytic system of claim 1, wherein the products include long-chain aliphatic hydrocarbons suitable for biofuel applications.

15. The method of claim 2, wherein the reaction products are isolated and characterized using MALDI-TOF mass spectrometry, IR spectroscopy, and NMR spectroscopy.

16. The method of claim 2, wherein the catalytic process operates continuously under ambient temperature and pressure without requiring pre-concentration of CO₂.

17. The catalytic system of claim 1, wherein the visible-light-driven process operates in a solid-state configuration, incorporating the catalytic complex into a photochemical reactor.

18. The method of claim 2, wherein the system can be scaled for industrial applications by employing a light-harvesting array to maximize photon absorption.
